(19) United States
(12) Patent Application Publication
(10) Pub. No.: US 2019/0321257 A1
(43) Pub. Date: Oct. 24, 2019

(54) SYSTEM FOR DELIVERING A VARIABLE PRESSURE FOR AN ENCLOSURE FOR TREATMENT OF A WOUND

(71) Applicant: DTAMEDICAL, Liuille (FR)

(72) Inventor: François DUFAY, Louille (FR)

(21) Appl. No.: 16/311,498

(22) PCT Filed: Jun. 15, 2017
(86) PCT No.: PCT/FR2017/051560

§ 371 Date: Dec. 19, 2018

(30) Foreign Application Priority Data
Jun. 20, 2016 (FR) 16/55720

(51) Int. Cl. A61H 33/14; A61H 35/00; A61G 10/02; A61H 33/02; A61K 33/00

(52) U.S. Cl. CPC A61H 33/14; A61H 35/006; A61G 10/02; A61H 33/02; A61K 33/00; A61H 2201/5092; A61H 2033/145; A61H 2201/0157; A61H 2201/165; A61H 2201/5071; A61H 2201/5082; A61H 2033/143

(57) ABSTRACT

A system (5) for generating a gaseous mixture for an enclosure (7) for treatment of a wound (3), comprising a generator (11) for generating the gaseous mixture and for delivering said gaseous mixture to the treatment enclosure (7) at a defined flow rate, the generator (11) of the gaseous mixture being designed to vary a molar fraction of dioxygen of said delivered gaseous mixture and to vary a molar fraction of water vapour of said delivered gaseous mixture, a control unit (39) which serves for controlling the delivered gaseous mixture and is provided with a processor (41) for receiving information relating to a pressure (P) inside the treatment enclosure (7) and/or designed to determine a flow rate of said gaseous mixture to be delivered in the treatment enclosure (7) in order to obtain an internal target pressure, the target pressure being defined by a time variation function of the pressure between a minimum pressure, corresponding to an ambient pressure outside the treatment enclosure (7), and a maximum pressure.

The present invention relates to a system for generating a gaseous mixture for an enclosure for treatment of a wound.

For wound treatment, the method is known of using a treatment enclosure that exposes the wound to a gaseous mixture, accelerating healing.

This arrangement is satisfactory in that the wound is maintained in a treatment area that has a controlled environment.

In practice, however, handling of the composition of the gaseous mixture and its mode of diffusion onto the wound can be difficult to implement.

In particular, even if the action of the components of the gaseous mixture alone is beneficial for healing of the wound, these components do not always allow effective stimulation of the blood and/or lymphatic circulation. Additional care may be necessary for healing.

The purpose of the present invention is to overcome all or some of the drawbacks mentioned above.

To this end, the present invention relates to a system for generating a gaseous mixture for an enclosure for treatment of a wound comprising;
a gaseous mixture generator for delivering said gaseous mixture to the treatment enclosure at a defined flow rate, the gaseous mixture generator being designed to vary a molar fraction of dioxygen of said delivered gaseous mixture and to vary a molar fraction of water vapor of said delivered gaseous mixture,
a control unit for controlling the delivered gaseous mixture provided with a processor for receiving data relating to a pressure inside the treatment enclosure and/or designed to determine a flow rate of said gaseous mixture to be delivered inside the treatment enclosure in order to obtain an internal target pressure,

the target pressure being defined by a time variation function of the pressure between a minimum pressure, corresponding to an ambient pressure outside the treatment enclosure, and a maximum pressure,

the processor also being designed to determine a time variation function of the molar fraction of dioxygen of the gaseous mixture to be delivered and a time variation function of the molar fraction of water vapor of the gaseous mixture to be delivered.

The system for generating a gaseous mixture thus makes it possible, by means of the time modulation of the pressure and the composition of the gaseous mixture, to expose the wound to an environment that favors its healing.

The lymphatic and blood circulation in the damaged tissues of the wound is thus stimulated. The lymphatic circulation also allows disposal of the waste material generated by the inflammation process required for healing of the wound.

Moreover, the variation of the pressure makes it possible to stimulate the tissues without damaging them, regardless of the surface to be treated; in a non-exhaustive manner, this surface is planar for a venous ulcer and three-dimensional for a diabetic foot.

The pressure management carried out by the generating system therefore makes it possible to treat various pathologies of acute or chronic wounds, including but not limited to wounds of the upper extremities, venous ulcers, chest wounds, abdominal wounds, pressure sores, and burns requiring pressure therapy.

According to one aspect of the invention, the gaseous mixture generator comprises a recirculating device configured to receive at least a part of the gaseous mixture having passed through the treatment enclosure and to incorporate it into the gaseous mixture delivered to the treatment enclosure.

The generating system is a semi-open or closed system that makes it possible to economize on certain resources such as dioxygen or water vapor.

According to one aspect of the invention, the recirculating device comprises a filter for filtering at least a part of the gaseous mixture having passed through the treatment enclosure. This arrangement makes it possible to limit recirculation of waste materials or organisms originating from the wound.

Preferably, the recirculating device comprises a dehumidifier and/or a humidifier. This arrangement makes it possible to vary the molar fraction of water vapor contained in the delivered gaseous mixture.

According to one aspect of the invention, the gaseous mixture generator is for delivering said gaseous mixture to the treatment enclosure at a defined relative humidity.

This arrangement makes it possible to regulate the relative humidity independently of the pressure and the molar fraction of dioxygen present in the gaseous mixture.

According to one aspect of the invention, the gaseous mixture generator is configured to deliver inside the treatment enclosure said gaseous mixture at a defined relative humidity.

According to one aspect of the invention, the processor is for receiving data relating to a relative humidity inside the treatment enclosure and/or is designed to determine a relative humidity of said gaseous mixture to be delivered inside the treatment enclosure in order to obtain an internal target relative humidity.

It is therefore possible to modulate the relative humidity of the delivered gaseous mixture and/or to regulate the relative humidity based on a value measured in the treatment enclosure or based on a value estimated for example by calculation or correlation with other known or measured values.

According to one aspect of the invention, the time variation function of the molar fraction of water vapor or of the relative humidity is a periodic function, and preferably, said periodic function is of the sinusoidal type, the triangular type, or the step type.

This arrangement allows regular variation for continuous treatment of the wound during a long period of several hours to several months.

According to one aspect of the invention, the gaseous mixture generator is for delivering said gaseous mixture to the treatment enclosure at a defined temperature.

This arrangement allows operation regardless of the conditions around the treatment enclosure, particularly ambient temperature. Indeed, if the enclosure is located in a room where the temperature is too low or too high, this is irrelevant. The wound continues to be exposed to the gaseous mixture at the optimum temperature for allowing rapid healing thereof.

According to one aspect of the invention, the gaseous mixture generator is configured to deliver said gaseous mixture inside the treatment enclosure at a defined temperature.

According to one aspect of the invention, the processor is for receiving data relating to a temperature inside the treatment enclosure and/or is designed to determine a temperature of said gaseous mixture to be delivered inside the treatment enclosure in order to obtain an internal target temperature.

According to one aspect of the invention, the processor is designed to determine a time variation function of the temperature of the delivered gaseous mixture.

The temperature of the gaseous mixture to be delivered can thus be determined and/or regulated. In particular, this arrangement allows a user to move around with the generating system and the treatment enclosure, for example inside, without having to make new adjustments.

Moreover, the properties of the material making up the enclosure are not limited to those of thermally insulating materials.

According to one aspect of the invention, said internal target pressure is between 28° C. and 43° C., and preferably between 28 and 40° C. The generating system thus makes it possible to expose the wound to different temperatures depending on the pathology being treated.

According to another aspect of the invention, said internal target pressure is less than 28° C.

According to one aspect of the invention, the processor is designed to determine a treatment program comprising at least one functioning phase according to a mode of functioning such as treatment of the inflammatory phase, the proliferative phase, or the epithelialization phase.

The treatment program is therefore defined according to the status of the wound. Even in the case of three modes of functioning, a greater number of treatment programs can be developed, as each program comprises the successive application of multiple functioning modes, each of specific duration.

According to one aspect of the invention, the time variation function of the pressure is a periodic function.

This arrangement allows the pressure applied to the skin to be alternated; this alternation has a therapeutic effect.

According to one aspect of the invention, said periodic function is of the sinusoidal type, the triangular type, or the step type.

According to one aspect of the invention, the gaseous mixture generator is configured to maintain the pressure inside the treatment enclosure constant for a specified period of time.

The wound can thus be maintained under pressure for a specified period of time. This arrangement is useful for example for attempting to limit certain complications of healing.

According to one aspect of the invention, the time variation function of the pressure comprises an increasing pressure phase and a decreasing pressure phase, with the duration of each phase depending on the desired therapeutic effect.

According to one aspect of the invention, a pressure maintenance phase follows the increasing pressure phase and precedes the decreasing phase.

According to one aspect of the invention, the target pressure is between 0 and 600 mmHg above the ambient pressure outside the treatment enclosure.

According to one aspect of the invention, the target pressure is between 0 and 300 mmHg above the ambient pressure outside the treatment enclosure.

According to one aspect of the invention, the target pressure is between 30 and 40 mmHg above the ambient pressure outside the treatment enclosure area, which has a therapeutic effect adapted to certain wound situations.

According to one aspect of the invention, the target pressure is between 45 and 60 mmHg above the ambient pressure outside the treatment enclosure. In particular, this arrangement is complementary to burn treatment and can be of therapeutic benefit in certain anatomical areas.

According to one aspect of the invention, the target pressure is between 200 and 300 mmHg above the ambient pressure outside the treatment enclosure. These pressures can be useful for treating severe conditions.

According to one aspect of the invention, the molar fraction of dioxygen is between 0 and 1.

According to one aspect of the invention, the processor is for receiving data relating to a molar fraction of dioxygen inside the treatment enclosure and/or for determining a molar fraction of dioxygen of said gaseous mixture to be delivered inside the treatment enclosure in order to obtain an internal target molar fraction of dioxygen.

It is therefore possible to modulate the molar fraction of dioxygen of the delivered gaseous mixture and/or to regulate the molar fraction of dioxygen based on a value that is measured in the treatment enclosure or estimated.

This arrangement makes it possible to vary the molar fraction of dioxygen to a significant degree in order to stimulate the wound.

According to one aspect of the invention, the time variation function of dioxygen comprises an increasing phase of the molar fraction of dioxygen and a decreasing phase of the molar fraction of dioxygen.

This variation of the molar fraction of dioxygen will favor the healing process.

According to one aspect of the invention, the time variation function of the molar fraction of dioxygen is a periodic function; in particular, said periodic function is of the sinusoidal type, the triangular type, or the step type.

According to one aspect of the invention, the gaseous mixture also comprises e.g. a molar fraction of dinitrogen and/or a molar fraction of carbon dioxide and/or another suitable gas such as nitrous oxide or other rare gases.

This arrangement allows the gaseous mixture generator to easily regulate the molar fraction of dioxygen and the molar fraction of water vapor. According to one aspect of the invention, the gaseous mixture comprises air.

According to one aspect of the invention, the processor is designed to determine a time variation function of the molar fraction of dinitrogen and/or a time variation function of the molar fraction of carbon dioxide and/or the other suitable gas of said gaseous mixture to be delivered inside the treatment enclosure.

This arrangement makes it possible to reduce the molar fraction of dioxygen and/or the molar fraction of water vapor in the gaseous mixture.

The molar fraction of carbon dioxide also makes it possible to regulate the pH of the gaseous mixture and thus obtain a pH adapted to the healing stage.

According to one aspect of the invention, the generating system comprises a device for adding an additional gas and/or an aerosol to the gaseous mixture delivered by the gaseous mixture generator, with said addition device being configured to nebulize said aerosol and/or vaporize and/or sublimate said additional gas in the gaseous mixture delivered by the gaseous mixture generator.

The simultaneous occurrence of variation of the molar fractions on the one hand and addition of an additional component on the other has a beneficial effect on the wound. The additional component is diffused in the form of an additional gas and/or an aerosol.

The term aerosol is understood to refer to a mass of fine liquid and/or solid particles. According to one aspect of the invention, the additional gas and/or the aerosol comprises an active ingredient for treating the wound.

This principle of diffusing the active ingredient simultaneously with variation of the composition of the gaseous mixture allows the healing process to be facilitated in an optimum manner.

This arrangement makes it possible to obtain a uniform gaseous mixture enriched with additional gas and/or aerosol. Preferably, the additional gas and/or the aerosol comprise an active ingredient suitable for exerting an effect on the wound.

Inside the treatment enclosure, the wound is therefore bathed in a gaseous mixture comprising an active ingredient. As the exposure to this active ingredient takes place continuously over a fairly long period ranging from several hours to several months, the wound is exposed to favorable conditions for healing.

According to one aspect of the invention, the control unit comprises a user interface provided with a user control and/or a feedback device comprising a screen and/or an indicator light.

The user is therefore able to influence regulation of the control unit.

In particular, the user can create, modify and/or select a time variation function of the pressure, the temperature, or the molar fraction of the components of the gaseous mixture.

According to one aspect of the invention, the processor comprises in its memory a plurality of time variation functions of the pressure, the temperature, and the molar fraction of the components of the gaseous mixture, the user control being configured to allow a user to select at least one time variation function to be applied by the generating system.

The generating system is therefore easy to use; the user must select the time variation function(s) corresponding to the status and type of the wound to be treated.

According to one aspect of the invention, the user interface comprises a locking function configured to limit the selection and/or creation and/or modification of at least one time variation function by means of a key. Said key is preferably an access code.

This arrangement makes it possible to define various management levels of the generating system; for example, a physician can have the access code in order to select any desired time variation function, while the number of accessible functions is limited for a nurse, and even more limited for the patient.

It is thus possible to ensure that the treatment will be properly followed while leaving a margin of adaptability of the device depending on the circumstances.

As for the feedback device, this device makes it possible to determine the feedback data originating from the enclosure, for example measured or estimated values. The estimated values may be derived from calculations or correlations carried out based on other known values or values measured in the enclosure or in the environment of the enclosure.

It is therefore possible for the physician to verify whether a treatment has been properly applied when he/she consults the history of the feedback data originating from the enclosure or the environment of the enclosure.

According to one aspect of the invention, the user interface comprises a plug for the transfer of data, for example a USB plug. This arrangement allows the user to transfer the entire history of the treatments carried out, for example in the form of a table of values. It is then possible to analyze the course of the wound based on these data.

Alternatively, the plug can be replaced by a transmitter for transmitting data according to a known protocol, for example a wire protocol or by radio waves.

According to one aspect of the invention, the system for generating a gaseous mixture comprises a measurement system configured to carry out transcutaneous oximetry of the wound and to transmit the measurement values to the processor.

This arrangement makes it possible to provide the processor with more data on the status of the wound by determining the real oxygenation status of the wound tissues.

Preferably, the measurement system is configured to measure a transcutaneous pressure for the dinitrogen and/or carbon dioxide at the level of the wound. Transcutaneous oximetry and transcutaneous pressure are also referred to by the abbreviations TcPO₂, TcPN₂, and TcPCO₂.

According to one aspect of the invention, the measurement system also comprises a capillaroscopy device and/or a wound temperature sensor. This arrangement makes it possible to provide the processor with more data on the status of the wound.

The more a wound is perfused by oxygenated arterial blood, the warmer it will be; the inflammation means that the arterial blood has brought all of the elements necessary for tissue repair or increasing the temperature.

According to one aspect of the invention, the measurement system is also configured to carry out measurement by fluorescence angiography or by measuring the arteriolar pressure of the wound, and/or comprises devices for laser doppler measurement and/or iontophoresis.

Various sensors can thus be configured on the treatment device for optimal identification of the status of the wound.

According to one aspect of the invention, the processor is configured to receive data relating to a dimension and/or a development phase of at least a part of a wound.

This arrangement allows the processor to adjust the mixing parameters such that treatment of the wound is optimal and adaptable. As this adjustment is carried out automatically by the processor, there is no risk of user error in selecting the settings.

According to one aspect of the invention, said data relating to the dimension and/or the development phase, for at least a part of the wound, comprise an area and/or a development phase, including an inflammatory phase, a proliferative phase and an epithelialization phase.

The size of the wound observed can thus be constant, and it can comprise several parts, the area and/or the development phase of which change.

This arrangement therefore allows the processor to adapt the time variation functions to the size of the wound and/or its various component parts. The treatment proposed by the generating system makes it possible not only to target a specified type of wound, but also to make adaptations to the size of the wound and/or its various component parts.

According to one aspect of the invention, the control unit comprises a sensor for determining said data relating to the dimension and/or the development phase of at least a part of the wound. Preferably, said sensor comprises a video camera.

This arrangement allows constant adaptation of the proposed treatment to the wound.

According to one aspect of the invention, the video camera is disposed on an arm of the generating system, with it being possible to move the video camera in order to capture an image of at least a part of the wound.

According to one aspect of the invention, the video camera is arranged in the treatment enclosure. This arrangement allows automated imaging, as the video camera is fixed with respect to the treatment enclosure.

According to one aspect of the invention, the sensor for determining said data relating to the dimension and/or the development phase of at least a part of the wound comprises at least two video cameras.

Preferably, the at least two video cameras are arranged in the treatment enclosure. This arrangement makes it possible to carry out a three-dimensional reconstruction of the wound in order to obtain more detailed data on the wound status.

According to one aspect of the invention, the user control is configured to allow a user to acquire, select, and/or modify said data relating to the dimension and/or the development phase.

This arrangement allows manual determination of the size and development of a wound. For example, this can be updated by the physician during consultation.

According to one aspect of the invention, the processor is configured to store in memory at least one piece of data relating to the dimension and/or the development phase of the wound. Preferably, the processor is configured to store in its memory a history of the changes in said piece of data.

According to one aspect of the invention, the processor comprises a treatment module designed to determine a development phase of all or part of the wound, the determination being carried out based on the data relating to a dimension and/or a development phase of at least a part of a wound.

This arrangement makes it possible to complete the visual analysis of the wound carried out by a physician; not only does the control unit make it possible to acquire data relating to a dimension and/or a development phase of the wound, but it also allows processing of these data in order to determine the development phase of all or part of the wound.

According to one aspect of the invention, the treatment module is designed to determine a contour of all or part of the wound and to assign, to an area delimited by said contour, a development phase, the assignment being carried out based on the data relating to a dimension and/or a development phase of at least a part of a wound.

This arrangement makes it possible to determine the contours of various parts of the wound according to their development phases.

This arrangement thus makes it possible not only to use the measured data, but also to deduce from them the characteristics of the wound by cross-tabulating these data.

According to one aspect of the invention, the contour is determined by a segmentation method of the watershed or regional growth type, said method being implemented by the treatment module.

The methods or algorithms applied by the treatment module are reliable and limit the resource calculation requirements of the processor with respect to the precision obtained. This allows favorable identification of the status of the wound.

According to another aspect of the invention, the treatment module is configured to compare the data relating to a dimension and/or a development phase of at least a part of a wound with a plurality of data on definitions of development phases for determining the development phase of all or part of the wound.

This arrangement makes it possible to dispense with the need for determining the contour. In fact, comparison with the definition data makes it possible to compare the wound as a whole with a definition of a known stage.

According to another aspect of the invention, the comparison is carried out according to a method of local binary patterns. Use of this method makes it possible to dispense with the segmentation step and thus to improve the robustness of the method.

According to one aspect of the invention, a development phase is defined by a plurality of descriptive elements, in particular the color and the texture of the area delimited by the contour.

This arrangement makes it possible to precisely define the development phase taking into account all of the successive stages of changes in the status of the wound.

According to one aspect of the invention, the comparison is carried out according to a kernel partial least squares regression or KPLS method, with said method being implemented by the treatment module and the result being a probability of belonging to a development phase.

This arrangement makes it possible to determine based on measured data the most probable status of the wound. The determination is thus rapid and reliable.

According to another aspect of the invention, the development phase is determined by a classification method, in particular software vector machine or deep learning.

This arrangement makes it possible to provide possible alternatives for identifying the development phase.

According to one aspect of the invention, the treatment module is configured to implement an initial learning phase during which a plurality of definition data are transmitted to the determination module, with each piece of data corresponding to a given development phase.

This arrangement allows calibration of the treatment module, with it thus being possible to adapt this calibration according to the type of material used for the measurements and/or according to the type of wound treated.

According to one aspect of the invention, the treatment module is designed to determine a control set point defining the time variation function of the pressure and/or the time variation function of the molar fraction of dioxygen.

The treatment module thus makes it possible, after the measured values have been analyzed, to determine by calculation the control set point best adapted to the status of the wound.

According to one aspect of the invention, the control set point defines for the gaseous mixture a relative humidity, a temperature, a molar fraction of water vapor, a molar fraction of dinitrogen, a molar fraction of carbon dioxide, and/or a fraction of additional gas or aerosol.

Preferably, the user interface is configured to allow user access in order to validate the control set point determined by the treatment module.

The control set point is thus to be considered a proposal made to the treating physician, who can then, if necessary, modify the control set point if his/her analysis of the status of the wound is different.

According to another aspect of the invention, the control unit is configured to automatically apply the control set point after it is determined. This arrangement makes it possible to automate treatment of the wound by adapting the treatment to the reaction of the wound.

According to one aspect of the invention, the treatment module is designed to determine at least two control commands, with a relevance level preferably being assigned to each control set point.

This arrangement allows the physician to select the treatment he/she feels is best for the wound.

According to one aspect of the invention, the generating system is configured to be attached to an infusion stand and/or a bedhead unit.

This arrangement makes it possible to easily move the generating system. The user treating his/her wound can therefore move with the generating system.

The ambulatory function is particularly useful for chronic wounds such as venous ulcers, diabetic foot, or arteritis. The treatment does not prevent the user from moving.

The present invention also relates to a treatment device comprising a generating system as described above and a treatment enclosure configured to be disposed on and/or around a body part of a user so as to create a treatment area.

According to one aspect of the invention, the treatment enclosure comprises an inlet for the gaseous mixture delivered by the generating system inside the treatment area.

According to one aspect of the invention, the treatment enclosure is configured to surround a body part, for example an upper extremity or a foot of the user, and comprises a sole allowing the user to walk. This arrangement is useful in the case of diabetic foot; the user becomes independent.

According to one aspect of the invention, the treatment enclosure comprises a contact surface capable of cooperating with the skin of a user, the contact surface being capable of fluidically isolating the external area.

According to one aspect of the invention, the contact surface is suitable for adhering to the skin, and the treatment enclosure preferably comprises a product allowing the wound to be maintained in a manner isolating it from the external environment, said product being disposed on said contact surface.

This arrangement makes it possible to fluidically isolate the treatment enclosure by causing the contact surface to adhere to the skin by means of an adhesive product.

According to one aspect of the invention, the treatment enclosure comprises a seal on which the contact surface is arranged. Preferably, the seal is composed of silicone.

According to one aspect of the invention, the seal comprises an internal chamber having openings communicating with the treatment area, the outlet and/or the inlet of the treatment area. The seal is thus maintained in position by the gaseous mixture penetrating into the internal chamber.

This arrangement makes it possible to improve the grip of the treatment enclosure around an extremity of the user.

According to one aspect of the invention, the treatment enclosure comprises an external envelope capable of assuming a defined shape when the generating system delivers the gaseous mixture, the envelope being capable of being deformed in response to a constraint imposed on its surface and of re-assuming the defined form when said constraint is removed.

This arrangement allows the wound to be massaged, because the constraint makes it possible to locally modify the pressure and thus locally deform the skin. From a medical standpoint, massaging the wound increases the possibility of exchanges between the gaseous mixture and the wound tissues and improves the blood and lymphatic circulation, which is useful for certain types of wounds.

This arrangement makes it possible to enhance the physiology of healing.

According to one aspect of the invention, the time variation function of the molar fraction of dioxygen and/or water vapor and/or carbon dioxide and/or dinitrogen and/or any other component of the air comprises an increasing phase of said molar fraction and a decreasing phase of said molar fraction.

According to one aspect of the invention, a maintenance phase of the molar fraction follows the increasing phase and precedes the decreasing phase.

In the present text, the delivered gaseous mixture can also be defined by a delivery pressure instead of the delivered flow rate. In this case, a partial pressure of the delivery pressure can be defined for each component. This partial pressure of a component is equivalent to the molar fraction of the component multiplied by the delivery pressure.

In any event, the invention will be clearly understood by means of the following description with reference to the attached schematic drawings, which show, as a non-limiting example, an embodiment of this system for generating a gaseous mixture.

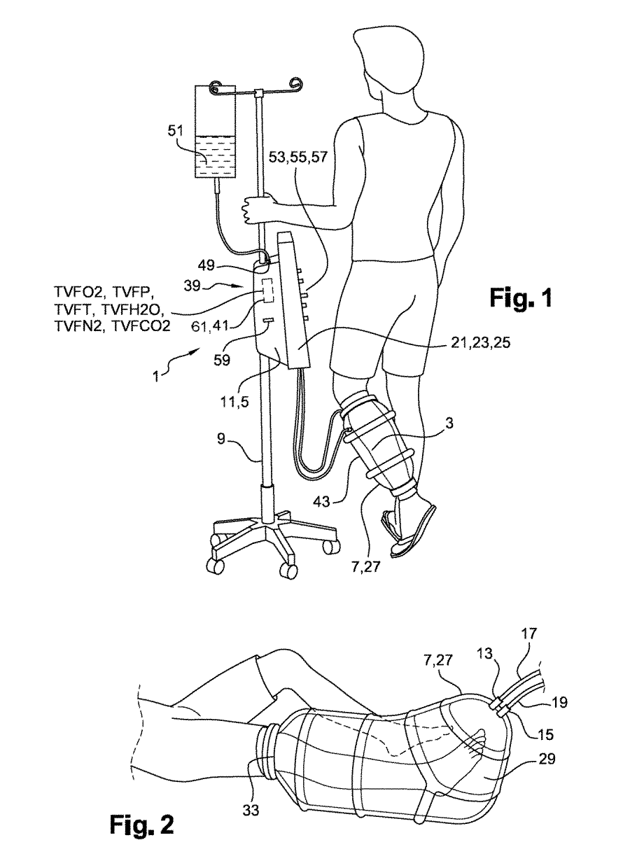

FIG. 1 is a perspective view of a treatment device for a wound and a user.

FIGS. 2 to 8 are perspective views of a treatment enclosure of the treatment device.

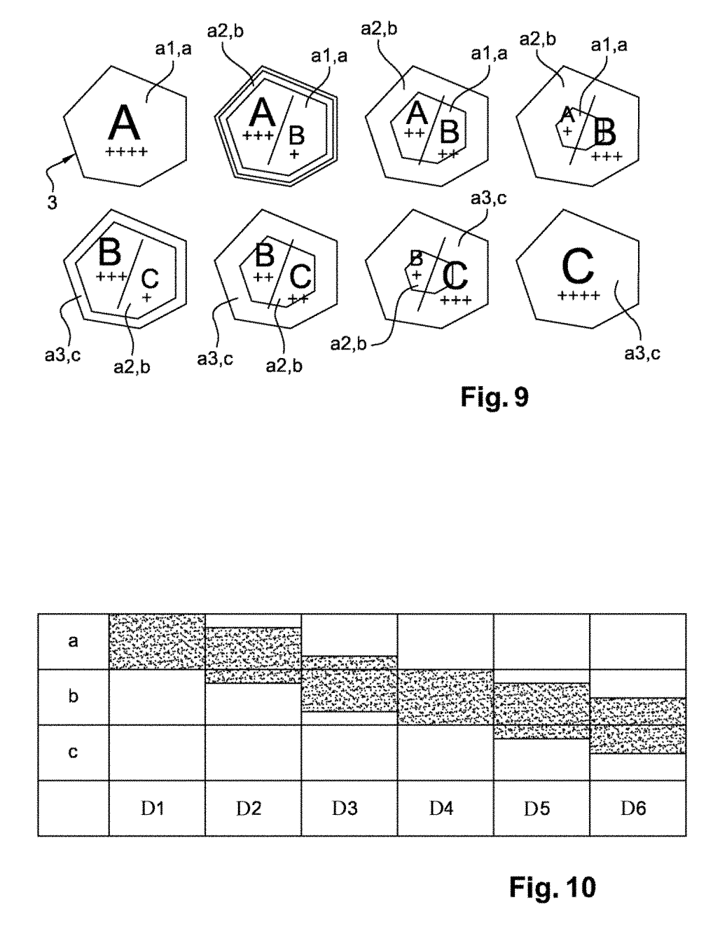

FIG. 9 is a diagram showing development phases of the wound.

FIG. 10 is a graphical representation of the development phases of the wound over time.

As shown in FIG. 1, a treatment device 1 of a wound 3 comprises a system 5 for generating a gaseous mixture and a treatment enclosure 7.

The generating system 5 is configured to be attached to an infusion stand 9. In addition, the generating system 5 is configured to be attached to a bedhead unit. The treatment device 1 can therefore be mobile or stationary according to the constraints of use.

The generating system 5 comprises a gaseous mixture generator 11 for delivering the gaseous mixture inside the treatment enclosure 7 at a defined flow rate.

For this purpose, and as shown in FIGS. 2 to 8, the treatment enclosure 7 is provided with an inlet 13 for the gaseous mixture originating from the gaseous mixture generator 11.

The treatment enclosure 7 also comprises an outlet 15. The delivered gaseous mixture therefore circulates inside the treatment enclosure 7.

The generating system 5 comprises an inlet line 17 connecting the gaseous mixture generator 11 to the inlet 13 of the treatment enclosure 7 and a return line 19 towards a recirculating device 21 of the generating system 5.

The recirculating device 21 is configured to receive at least a part of the gaseous mixture that has passed through the treatment enclosure 7 and to incorporate it into the delivered gaseous mixture.

It is thus possible not to recirculate the gaseous mixture, for example if it is necessary to change the composition of the gaseous mixture or to recirculate part or all of the gaseous mixture in order to economize on the components of the gaseous mixture.

The recirculating device 21 also comprises a filter 23 capable of removing from the gaseous mixture waste materials originating from the wound 3 and a humidifier 25 for adjusting a relative humidity inside the treatment enclosure.

The treatment enclosure 7 comprises an external envelope 27 capable of assuming a defined shape when the generating system 5 delivers the gaseous mixture.

The defined shape is obtained by deployment of the external envelope 27, because a pressure P inside the treatment enclosure 7 is greater than an ambient pressure outside the treatment enclosure 7 under the conditions of use.

In the present text, therefore, when reference is made to a pressure P inside the treatment enclosure 7 under functioning conditions, this pressure P is expressed as a relative pressure with respect to the external ambient pressure.

The external envelope 27 is also capable of being deformed in response to a constraint placed on it and of reassuming the defined form when said constraint is removed.

This arrangement makes it possible to indirectly massage the wound 3 by pressing on the outer envelope. In fact, this pressure locally modifies the pressure P inside the treatment enclosure 7 without requiring that the wound 3 or the skin around the wound 3 be touched.

The treatment enclosure 7 is configured to be disposed on and/or around a body part of a user so as to create a treatment area 29 in which the gaseous mixture circulates.

The treatment enclosure 7 comprises a contact surface 31 capable of conforming to the skin of the user, the contact surface 31 being capable of fluidically isolating the treatment area 29 of the treatment enclosure 7 from the exterior.

The contact surface 31 can be arranged in a silicone seal 33 of the treatment enclosure, as shown in FIGS. 2, 3, 5, 6 and 8. The treatment enclosure 7 also comprises, either instead of or in addition to the silicone seal 33, an adhesive product 35 disposed on said contact surface 31. This is shown by way of example in FIGS. 4, 6 and 7.

Optionally, the silicone seal 31 can comprise an internal chamber having openings communicating with the treatment area and the outlet of the treatment area as shown in FIGS. 5 and 6.

The fact that the internal pressure P is greater than the ambient pressure therefore does not mean that the gaseous mixture will leak toward the exterior.

In FIG. 8, the generating system 5 also comprises straps 37 for maintaining the treatment enclosure 7 in position.

The generating system 5 comprises a control unit 39 provided with a processor 41 designed to control the gaseous mixture generator 11 and define a flow rate, a temperature, and a composition of the gaseous mixture to be delivered.

In addition, the gaseous mixture generator 11 comprises a tank for each component of the gaseous mixture or is configured to be attached to one or a plurality of external bottles.

The gaseous mixture comprises a molar fraction of dioxygen O₂ and a molar fraction of water vapor H₂O. The gaseous mixture can also comprise a molar fraction of dinitrogen N₂, a molar fraction of carbon dioxide CO₂, and/or a molar fraction of components of the air other than those mentioned above.

The processor is designed to determine a time variation function of the molar fraction of dinitrogen TVFN₂ and/or a time variation function of carbon dioxide TVFCO₂ of said gaseous mixture to be delivered inside the treatment enclosure.

The control unit 39 can comprise sensors 43 for determining an internal pressure P, an internal relative humidity RHH₂O, or a temperature T inside the treatment enclosure 7.

These sensors 43 make it possible to respectively transmit to the processor 41, according to a wire communication protocol or by radio waves, a piece of data relating to the current internal pressure P, the current relative humidity RHH₂O, and the current internal temperature.

One of the sensors 43 can also be a video camera 65 for transmitting a piece of data relating to a dimension a1, a2, a3 and to a development phase a, b, c of the wound or at least a part of the wound.

A dimension a1, a2, a3 is understood to refer to an area and a development phase a, b, c to refer to one of the three following phases; inflammatory a, proliferative b, or epithelialization c.

It is also understood that a wound can be defined by a piece of data relating to several parts of a wound, with each of the parts being defined by a dimension a1, a2, a3 and a development phase a, b, c. In a way, the wound is modelled in several parts, each being defined by an area having its own development phase, as shown in FIG. 9.

All of these data are input data for the processor 41, which can define the composition, the flow rate, and the temperature of the gaseous mixture to be delivered so that a treatment program is applied to the wound 3.

The processor 41 is configured to define a target temperature T, a target relative humidity RHH₂O, and a target pressure P inside the treatment enclosure 7 and to modify accordingly the composition of the gaseous mixture delivered inside the enclosure, its temperature, and its flow rate.

These target values can be constant, but may also vary based on time, for example in the form of periodic functions of the sinusoidal, triangular, or step type.

The processor 41 is thus configured to define a time variation function of the pressure inside the enclosure TVFP, with the pressure P always being greater than the ambient pressure outside the treatment enclosure.

The function of variation of the pressure TVFP makes it possible to improve the blood circulation and depends on the type of wound 3.

The processor 41 is also configured to define a time variation function of the molar fraction of dioxygen TVFO₂ of the delivered gaseous mixture, a time variation function of the molar fraction of water vapor TVFH₂O of the delivered gaseous mixture, and a time variation function of the temperature TVFT inside the treatment enclosure 7. FIG. 10 shows the course of development of a wound according to the phases a, b, and c defined in FIG. 9 over six days, D1 to D6. The letters A, B and C are the modes of functioning applied successively to the wound according to phases a, b, and c.

When the wound comprises several parts of different phases, for example a and b, a treatment program comprises a first part according to one mode then a second part according to another mode, for example A and B. The duration of each part can also be modulated.

The generating system 5 also comprises an addition device 49 for adding to the mixture an additional gas and/or an aerosol 51 in nebulized, vaporized, or sublimated form.

The additional gas and/or the aerosol 51 can comprise an active ingredient for the treatment of the wound 3 or any type of product having a positive effect on the healing of the wound 3. For example, this can be an aqueous solution comprising active ingredients for treatment of the wound.

The processor 41 is also configured to vary the quantity of additional gas and/or aerosol 51 in the gaseous mixture.

The generating system 5 also comprises a user interface 53 provided with a user control 55 configured to allow the user either to select a treatment program or to manually modify a time variation function TVF.

Each modification is made possible by entering a key of the access code type in order to override a default locking of the user control 53. Depending on the key used, the user will have access to a greater or lesser number of options.

One key can thus be defined for a physician, another for a nurse with fewer possible settings, and another for a user provided only with limited access to the settings.

The user interface 51 can also be used to define the data relating to the dimension a1, a2, a3 and the development phase a, b, c of parts of the wound 3, for example if the generating system does not comprise a video camera 65.

The user interface 53 also comprises a feedback device 57 provided with a screen and/or an indicator light to allow the user to consult the settings of the time variation functions TVF of pressure and the data readings from the sensors 43.

The user interface 53 also comprises a data transfer plug 59, for example a USB plug, for transferring data concerning the settings or a history of measurements of the sensors 43 and data concerning the treatment programs previously applied by the user. The transferred data can be in the form of a table of values.

Alternatively, the plug can be replaced by a transmitter for transmitting data according to a known protocol, for example a wire protocol or by radio waves,

The treatment device 1 described has the advantage of being portable; the user can therefore move around with it; it can be used for extremely long periods, such as several days or weeks, without hindering the user's life.

This treatment device 1 also makes it possible to permanently adapt the desired treatment to the type of wound and course of development thereof.

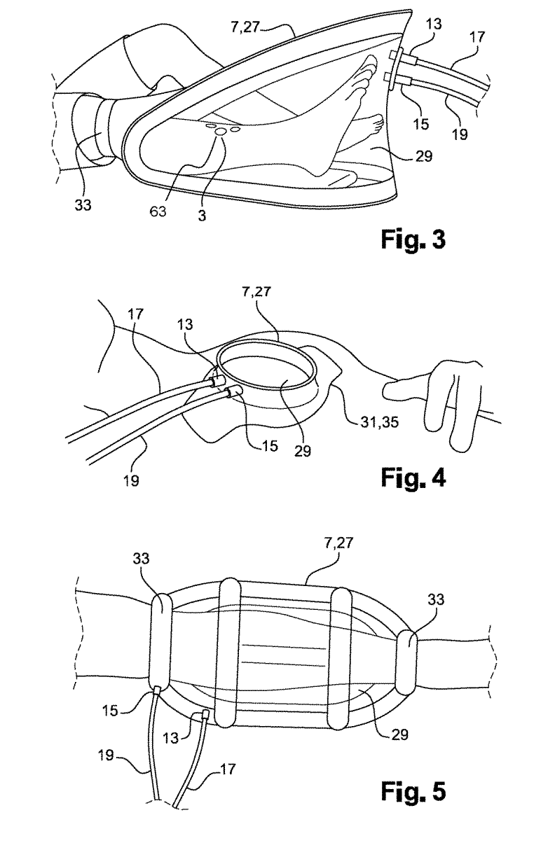

As shown in FIGS. 1 and 3, the processor 41 comprises a treatment module 61 designed to determine at least one contour 63 of all or part of the wound 3.

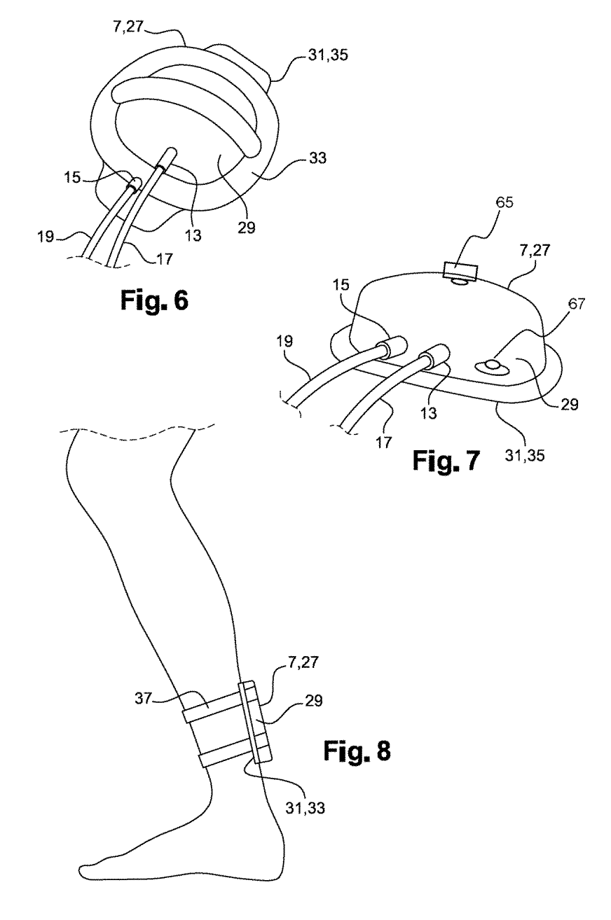

The treatment module 61 is thus configured to utilize the image provided by the video camera 65 as shown in FIG. 7 for determining one or more contours 63 of the wound 3 based on the number of distinct parts of the wound 3.

The treatment module 61 is also configured to allocate a development phase a, b, c to the area or each area delimited by a contour 63. This determination can be carried out by a segmentation method of the watershed or regional growth type.

Alternatively, a method of local binary patterns can be used by the treatment module 61.

When the area(s) has/have been determined, the treatment module 61 allocates to each of the area(s) an individual development phase a, b, c. A development phase a, b, c is defined by a plurality of descriptive elements such as a color and/or a texture.

The development phase a, b, c is determined by a kernel partial least squares regression or KPLS method, the result being a probability of belonging to a development phase a, b, c.

Alternatively, a classification method of the software method machine or deep learning type can be used by the treatment module.

The treatment module 61 is also configured to implement an initial phase, i.e. prior to functioning, during which a plurality of data relating to development phases is transmitted to the determination module, with there being a correspondence with a given development phase a, b, c for each piece of data.

In addition, and as shown in FIG. 7, the system 5 for generating a gaseous mixture comprises a measurement system 67 configured to carry out transcutaneous oximetry of the wound 3 and configured to transmit the measurement values to the processor 41.

The purpose is to inform the processor 41 of the status of the wound 3 by determining the real oxygenation status of the tissues of the wound 3. Transcutaneous oximetry is also referred to by the abbreviation TcPO₂.

According to the variant embodiments, the measurement system 67 can also comprise a capillaroscopy device and/or a wound temperature sensor.

The treatment module 61 is thus configured to centralize the measurement data in order to utilize them. The treatment module 61 is also designed to determine a treatment set point based on all of these data.

The control set point defines the time variation function of the pressure TVFP and/or the time variation function of the molar fraction of dioxygen TVFO₂.

The treatment module 61 thus makes it possible, after the measured values have been analyzed, to determine by calculation the control set point best adapted to the status of the wound 3.

The control set point defines for the gaseous mixture a relative humidity, a temperature T, a molar fraction of water vapor H₂O, a molar fraction of dinitrogen N₂, a molar fraction of carbon dioxide CO₂, and/or a fraction of additional gas or aerosol 51.

The user interface 53 is configured to allow the user to validate the control set point determined by the treatment module 61.

The control set point is thus to be considered a proposal made to the treating physician, who can then, if necessary, modify the control set point if his/her analysis of the status of the wound 3 is different.

It can therefore be seen that the treatment module 61 considers the real status of the wound and its course of development in order to determine the most suitable treatment. The fact that the interface 53 is configured to propose a treatment allows the physician to gain time if he/she realizes that his/her analysis is identical.

In such a case, the physician only needs to validate the proposal of the treatment module 61.

It is obvious that the invention is not limited only to the embodiment of this system for generating a gaseous mixture, which is described above by way of example, but includes all variant embodiments.

1. A system for generating a gaseous mixture for an enclosure for treatment of a wound comprising;
a gaseous mixture generator for delivering said gaseous mixture to the treatment enclosure at a defined flow rate, the gaseous mixture generator being designed to vary a molar fraction of dioxygen (O2) of said delivered gaseous mixture and to vary a molar fraction of water vapor (H2O) of said delivered gaseous mixture,
a control unit for controlling the delivered gaseous mixture provided with a processor for receiving data relating to a pressure (P) inside the treatment enclosure and/or designed to determine a flow rate of said gaseous mixture to be delivered inside the treatment enclosure in order to obtain an internal target pressure (P),
the target pressure (P) being defined by a time variation function of the pressure (TVFP) between a minimum pressure, corresponding to an ambient pressure outside the treatment enclosure, and a maximum pressure,
the processor also being designed to determine a time variation function of the molar fraction of dioxygen (TVFO2) of the gaseous mixture to be delivered and a time variation function of the molar fraction of water vapor (TVFH2O) of the gaseous mixture to be delivered.

2. (canceled)

3. The system of claim 1, wherein the gaseous mixture generator is for delivering said gaseous mixture to the treatment enclosure at a defined temperature (T).

4. The system of claim 1, wherein the time variation function of the pressure (TVFP), the molar fraction of dioxygen (TVFO2), the molar fraction of water vapor (TVFH2O), or temperature (T) inside the treatment enclosure is a periodic function.

5. The system of claim 4, wherein the periodic function is sinusoidal, triangular, or step.

6. The system of claim 1, wherein the gaseous mixture also comprises a molar fraction of dinitrogen (N2) and/or a molar fraction of carbon dioxide (CO2) and/or another suitable gas.

7. The system of claim 1, wherein the control unit comprises sensors for determining an internal pressure (P), an internal relative humidity (RHH2O), molar fraction of dioxygen TVFO2, or a temperature (T) inside the treatment enclosure.

8. The system of claim 7, comprising a measurement system configured to carry out transcutaneous oximetry of the wound and configured to transmit the measurement values to the processor.

9. The system of claim 7, wherein the measurement system also comprises a capillaroscopy device and/or a wound temperature sensor.

10. The system of claim 1, wherein the processor is configured to receive data relating to a dimension (a1, a2, a3) and/or to a development phase (a, b, c) of at least a part of a wound.

11. The system of claim 10, wherein the control unit comprises a sensor for determining said data relating to the dimension (a1, a2, a3) and/or the development phase (a, b, c) of at least a part of the wound.

12. The system of claim 10, wherein the processor comprises a treatment module designed to determine a development phase (a, b, c) of all or part of the wound, the determination being carried out based on the data relating to a dimension (a1, a2, a3) and/or a development phase (a, b, c) of at least a part of a wound.

13. The system of claim 12, wherein the treatment module is designed to determine a contour of all or part of the wound and to assign, to an area delimited by said contour, a development phase (a, b, c), the assignment being carried out based on the data relating to a dimension (a1, a2, a3) and/or a development phase (a, b, c) of at least a part of a wound.

14. (canceled)

15. The system of claim 12, wherein the treatment module is configured to compare the data relating to a dimension (a1, a2, a3) and/or a development phase (a, b, c) of at least a part of a wound with a plurality of data on definitions of development phases for determining the development phase (a, b, c) of all or part of the wound.

16. The system of claim 12, wherein the treatment module is designed to determine a control set point defining the time variation function of the pressure (TVFP) and/or the time variation function of the molar fraction of dioxygen (TVFO2), and/or a relative humidity, a temperature T, a molar fraction of water vapor H2O, a molar fraction of dinitrogen N2, a molar fraction of carbon dioxide CO2, and/or a fraction of additional gas or aerosol.

17. A treatment device comprising a generating system of claim 1 and a treatment enclosure configured to be disposed on and/or around a body part of a user so as to produce a treatment area.

18. A method of treating a wound, comprising
arranging a treatment chamber to a portion of a human's body comprising a wound;
delivering a gas mixture at a given flow rate into the treatment chamber; and
modulating target pressure (P) and composition of the gas mixture.

19. The method of claim 18, further comprising defining said target pressure (P) by a time variation function of the pressure (TVFP) between a minimum pressure, corresponding to an ambient pressure outside the treatment enclosure, and a maximum pressure.

20. The method of claim 18, further comprising determining the time variation function of the pressure (TVFP), the molar fraction of dioxygen (TVFO2), the molar fraction of water vapor (TVFH2O), or temperature (T).

21. The method of claim 18, further comprising delivering the gaseous mixture to the treatment chamber at a defined temperature (T).

22. The method of claim 18, wherein the time variation function of the pressure (TVFP), the molar fraction of dioxygen (TVFO2), the molar fraction of water vapor (TVFH2O), or the temperature (T) is a periodic function.

23. The method of claim 22, wherein the periodic function is sinusoidal, triangular, or step.

24. The method of claim 18, further comprising determining the target internal pressure (P), an internal relative humidity (RHH₂O), or a temperature (T) inside the treatment chamber by sensors comprised by a control unit.

25. The method of claim 18, wherein the gaseous mixture also comprises a molar fraction of dinitrogen (N2) and/or a molar fraction of carbon dioxide (CO2) and/or another suitable gas.

26. The method of claim 18, further comprising measuring transcutaneous oximetry of the wound and transmitting the measurement values to a processor.

27. The method of claim 18, further comprising measuring capillarity and/or wound temperature, said measuring made by a sensor.

28. The method of claim 18, further comprising determining data relating to a dimension (a1, a2, a3) and/or the development phase (a, b, c) of at least a part of the wound using a sensor comprised by a control unit.

29. The method of claim 24, further comprising sending data relating to the dimension (a1, a2, a3) and/or to the development phase (a, b, c) of at least a part of a wound to a configured processor.

30. The method of claim 24, further comprising determining a development phase (a, b, c) of all or part of the wound, the determining being carried out based on the data relating to a dimension (a1, a2, a3) and/or a development phase (a, b, c) of at least a part of a wound.

31. The method of claim 18, further comprising determining a contour of all or part of the wound and assigning an area delimited by said contour, the assigning being carried out based on the data relating to a dimension (a1, a2, a3) and/or a development phase (a, b, c) of at least a part of a wound.

32. The method of claim 18, further comprising comparing data relating to a dimension (a1, a2, a3) and/or a development phase (a, b, c) of at least a part of a wound with a plurality of data on definitions of development phases for determining the development phase (a, b, c) of all or part of the wound.

33. The method of claim 18, further comprising determining a control set point defining the time variation function of the pressure (TVFP) and/or the time variation function of the molar fraction of dioxygen (TVFO2) and/or), and/or a relative humidity, a temperature T, a molar fraction of water vapor H₂O, a molar fraction of dinitrogen N₂, a molar fraction of carbon dioxide CO₂, and/or a fraction of additional gas or aerosol.

34. The method of claim 18, wherein the treatment chamber is configured to be disposed on and/or around a body part of a user so as to produce a treatment area.
